(19) United States
(12) Patent Application Publication
(10) Pub. No.: US 2018/0110724 A1
(43) Pub. Date: Apr. 26, 2018

(54) FILM DOSAGE FORM WITH MULTIMODAL AND PARTICLE SIZE DISTRIBUTIONS

(71) Applicant: Intelgenx Corp., St-Laurent (CA)

(72) Inventors: Horst G. Zerbe, Hudson (CA); Nadine Paiement, St-Laurent (CA); Cormac Long, Parsippany, NJ (US); Rodolphe Obeid, Pierrefonds (CA)

(73) Assignee: Intelgenx Corp., St-Laurent (CA)

(21) Appl. No.: 15/848,819

(22) Filed: Dec. 20, 2017

Related U.S. Application Data
(63) Continuation-in-part of application No. 15/426,149, filed on Feb. 7, 2017.
Application No. 15/426,149 is a continuation-in-part of application No. 12/963,132, filed on Dec. 8, 2010.
(60) Provisional application No. 61/267,626, filed on Dec. 8, 2009.

(51) Int. Cl. A61K 9/00; A61K 9/14; A61K 9/70; A61K 31/4985; A61K 31/4709; A61K 31/58; A61K 31/19; A61K 31/40; A61K 31/34; A61K 31/496; A61K 31/439; A61K 31/553; A61K 31/353; A61K 47/14

(52) U.S. Cl. CPC A61K 9/006; A61K 9/14; A61K 9/7007; A61K 31/4985; A61K 31/4709; A61K 31/58; A61K 47/14; A61K 31/40; A61K 31/34; A61K 31/496; A61K 31/439; A61K 31/553; A61K 31/353; A61K 31/19

(57) ABSTRACT

Oral film dosage forms that provide improved solubilization and stabilization of an active ingredient in particle form include at least one primary crystallization inhibitor in an amount that inhibits growth and/or agglomeration of the active ingredient, a polyoxyethylated fatty acid glycerides in an amount that further enhances inhibition of crystallization, growth and agglomeration of the particles of the pharmaceutically active ingredient; and at least one plasticizer present in an amount that is effective to increase flexibility and elasticity of the film dosage form.

### CROSS-REFERENCE TO RELATED APPLICATIONS

This application claims benefit under 35 U.S.C. Section 120 to U.S. application Ser. No. 15/426,149, filed Feb. 7, 2017, titled SOLID ORAL FILM DOSAGE FORMS AND METHODS FOR MAKING SAME, which is a continuation-in-part of U.S. application Ser. No. 12/963,132, filed Dec. 8, 2010, and which claims benefit under 35 U.S.C. Section 119(e) of provisional application Ser. No. 61/267,626, filed Dec. 8, 2009, the entire contents of which are hereby incorporated by reference in their entireties.

### FIELD OF THE DISCLOSURE

This invention relates to solid oral pharmaceutical film dosage forms and more particularly to buccal and/or sublingual oral dosage forms comprised of at least one pharmaceutically active ingredient present as a stabilized plurality of particles.

### BACKGROUND OF THE DISCLOSURE

An oral film is a solid oral dosage form containing at least one water soluble polymer in combination with other acceptable ingredients and can provide therapeutic, nutritional and/or cosmetic effects. The polymeric matrix carrying the pharmaceutical, nutritional and/or cosmetic ingredient(s) is molded in a thin layer of variable area and shape. In contrast to conventional oral dosage forms, the administration of an oral film does not require water. A preferred site of administration is the buccal cavity. The solid oral dosage film can be placed on the tongue, on the cheek pouch, under the tongue or in the inner labial mucosa. The film is designed to deliver a drug in a manner that facilitates absorption of the drug. Oral film technology may be the preferred solid dosage option when aiming for a rapid onset of action and avoidance of the ‘first-pass effect’ (hepatic metabolism). It can also be used when compliance of the patient is an issue and/or concern. Pediatric and geriatric patients, or those with swallowing issues, will benefit the most through the use of orally disintegrating film technology, and oral film dosage forms will be of particular convenience when a discrete administration is preferred.

The pharmaceutically employed oral film is formulated to exhibit prompt hydration followed by a rapid or controlled dissolution/disintegration upon administration into the oral cavity. Upon administration and dissolution, the patient should not feel any discomfort during and/or immediately after its dissolution. Film forming polymers of common pharmaceutical use are water-soluble or water dispersible polymers that conform to the required properties, including, but not limited to, film instant hydration potential, mucoadhesion and solubility over time.

There are many major difficulties and challenges associated with the manufacture of oral film dosage forms ranging from brittleness, tackiness, the hygroscopic nature and potential lack of homogeneity within the dosage form. Ideal physical characteristics of the oral film include dosage uniformity throughout, adequate flexibility and tensile strength to facilitate processing, handling, and packaging of the film in a consumer-friendly form. All of those attributes should also be stable in time. Attaining ideal conditions for one characteristic usually comes at the expense of other, often equally important, properties, resulting in a necessary compromise in various properties to achieve a working film dosage form. Therefore, the main challenges and obstacles encountered when using oral film technology as a pharmaceutical delivery vehicle are due to the very properties upon which oral film technology is based. For example, challenges are encountered when attempting to provide an oral dosage as a film exhibiting a high content of liquid ingredients (0.5-35% wt/wt), and high drug loading in a matrix which is formulated as a very thin (typically under 80 micron) and continuous, yet flexible film layer.

An important requirement of drug delivery technology is the formulation of a delivery system that is capable of achieving a desirable release profile for the ever increasing number of active pharmaceutical ingredients with limited to poor water solubility. There are many conventional approaches for increasing the degree of solubilization of poorly soluble drugs including formation of ionizable molecules, use of a molecule in its amorphous state, pH adjustment and the development of co-solvent systems. However, these approaches can often be inadequate or inappropriate due to potential stability concerns. Particle size reduction has been a non-specific formulation approach that can be applied to almost any drug to enhance solubility. Due to greatly enhanced surface area obtained in this way, the dissolution rate and the bioavailability of poorly water-soluble drugs are expected to be high. Once the solid dispersion is exposed to aqueous media and the carrier dissolved, the drug is released as very fine, colloidal particles which can dissolve and be absorbed more rapidly than larger particles.

The increase in surface area results in a significant increase in surface energy leading to greater solubilization. However, the increase in surface energy is thermodynamically unfavorable and reagglomeration or crystallization/recrystallization of the particles is thermodynamically preferred resulting in a loss in the solubility of the material due to particle growth, and leading to decreased bioavailability. A preferred mechanism of stabilization of the reduced particles, for solid dosage forms, is physical stabilization of the particles through the dispersion of the particles on suitable polymers such as polyvinyl pyrrolidone, hydroxypropyl cellulose, hydroxypropylmethyl cellulose. This approach is often inadequate and leads to agglomeration and/or crystallization/recrystallization over time.

The key determinate properties in making an oral dosage film are the very particular features that facilitate the aggregation and/or crystallization to occur in an oral film relative to a classical solid dispersion (e.g. granulation, pellets, etc.). Granules, pellets, or free flowing powder can be tableted or encapsulated. The amount of water or any liquid ingredient in a tablet or capsule oral dosage form is typically less than 5%. The active ingredient is finely dispersed (sometimes down to a molecular level size) and is in very close contact with long chain or highly viscous polymers that physically limit reagglomeration of the active ingredient. However, these techniques are not suitable for the production of oral films characterized by a physical continuity of the matrix and a high level of liquid ingredients necessary to impart flexibility and tensile strength to the film. The resulting chemical environment allows the drug molecules a certain freedom to move and aggregate at a greater rate relative to other types of solid oral dosage forms. Reducing the amount of ingredients that impart flexibility to the oral film is undesirable, as it would result in a rigid matrix with reduced tensile strength and that is difficult to manufacture on a large scale. The recrystallization, agglomeration and/or aggregation phenomena must be avoided to maintain high drug bioavailability and to prevent an undesirable change in the physical characteristics of the film (strength, appearance, homogeneity, stability, etc).

A homogenous and stable distribution of the drug in the film matrix is of primary concern when developing an oral film for buccal delivery of a pharmaceutically active ingredient. Any increase in particle size due to aggregation and/or crystallization of the particles must be avoided to enhance transmucosal absorption and to limit the gastrointestinal absorption upon disintegration of the dosage form. It is well known that within the buccal cavity the amount of biological fluids (saliva) available for the solubilization of a drug is very limited as compared with the gastrointestinal fluids. Therefore, any process promoting faster dissolution of the active ingredient is generally desirable, but increases the need for maintaining stability of the pharmaceutically active ingredient. In particular, stabilization of the reduced particle size is desired to facilitate effective transmucosal absorption. If the active ingredient were to agglomerate or to crystallize within the dosage form, its solubility will, correspondingly, decrease and will result in the active ingredient being swallowed with the saliva.

Another characteristic in determining the resistance of the drug to reagglomeration within films is the extremely thin physical continuity of the matrix which provides minimal physical resistance to particle migration, and makes it difficult to prevent reagglomeration of the pharmaceutically active ingredient. Further concern arising from conventional particle size reduction techniques is the increase in the susceptibility of the active to degradation due to the increase in available surface area.

The prior art does not fully address the difficulty associated with preparing a pharmaceutical oral film capable of delivering a film dosage form with stabilized increased solubility and enhanced bioavailability while maintaining essential film characteristics.

### SUMMARY OF THE INVENTION

The solid dosage form described is an oral film for delivery of pharmaceutical, nutraceutical or cosmetic ingredients, with buccal delivery preferred. The film possesses a prompt or fast hydration potential, rapid or controlled dissolution and a stabilized increased water solubility of the active ingredient, thereby delivering the active ingredient available for immediate enhanced local absorption and consequently limiting loss or absorption later in the gastrointestinal route. The disclosed dosage forms provide among other things, improved delivery systems for solubilizing and stabilizing a plurality of pharmaceutically active ingredient particles in an effective particle size range that exhibit enhanced chemical stability, pharmaceutical formulations exhibiting improved bioavailability and/or absorption of pharmaceutically active ingredients when administered, and/or dosage forms for administration of pharmaceutically active ingredients achieved by the use of a combination of crystallization inhibitors, which together can maintain the active ingredient in a molecular dispersion within the polymeric film matrix. A method of oral film manufacture is also disclosed.

The disclosed solid dosage form is an oral film for delivery of an active ingredient present as a plurality of effectively stabilized particle sizes. The film has a controlled dissolution profile tailored to the inherent properties of the active to result in a given desired absorption and bioavailability while minimizing potentially hazardous early Cmax often associated with side effects. The oral film disclosed herein is designed for buccal delivery, enteral delivery or a combination of both. The oral film having a plurality of effectively stabilized particle size distributions generally allows modulation of the rate of absorption and bioavailability of the active ingredient.

The disclosed oral film dosage form maintains a plurality of active ingredient particles in an effective particle size range to maintain reduced structural order, and/or modulate solubility and bioavailability of the active. The film comprises a pharmaceutically poorly water soluble active ingredient in the form of amorphous particles having a first particle size distribution and a first D90 and a second particle size distribution having a second D90 wherein a difference between the second D90 and the first D90 is between 1 to 4 times the first D90.

The disclosed oral film dosage form maintains a plurality of active ingredient particles in an effective particle size range to maintain reduced structural order, and/or modulate solubility and bioavailability of the active. The film comprises at least one pharmaceutically highly water soluble active ingredient in the form of amorphous particles having a first and a second particle size distribution, the first particle size distribution having a first D90 and the second particle size distribution having a second D90, wherein a difference between the first D90 and the second D90 is between 1 to 9 times the first D90.

### DETAILED DESCRIPTION OF PREFERRED EMBODIMENT

The improved pharmaceutical oral dosage forms can comprise a pharmaceutically active ingredient, a primary polymeric crystallization inhibitor, a liquid crystallization inhibitor, a plasticizer, a penetration enhancing substance, a surfactant, a sweetening agent, a flavor, a flavor enhancer, an antioxidant, a starch and a colorant. The terms “a” and “an” as used herein should be construed to mean “at least one” or “one or more” unless otherwise indicated.

Unless otherwise noted, terms in this specification are intended to have their ordinary meaning in the relevant art.

The disclosed dosage forms can be used for delivery of a wide range of pharmaceutically active ingredients. According to some embodiments, the particle size and particle size distribution are synergistically stabilized by at least one primary crystallization inhibitor and at least one liquid crystallization inhibitor. The combination of the stabilization effect of each inhibitor on particle growth is greater than the sum of their individual stabilizing effects in the solid oral film dosage forms.

The term “liquid crystallization inhibitor” refers to any substance that exists in a liquid state at a temperature of about 37° C., and that in combination with the primary crystallization inhibitor or inhibitors, enhances the prevention and/or reduction of the rate of crystallization and/or agglomeration of the active substance or inhibits the growth of structural order (e.g., crystallization) of the active(s) in the film matrix over time and is mixable and/or compatible with the other excipients forming the film blend. The liquid crystallization inhibitor is present in the formulation in an amount that is effective for enhancing the prevention and/or reduction of crystallization and/or agglomeration of the active ingredient, and generally ranges from about 0.5% to 19% of the mass of the film dosage form. Certain non-limiting examples of liquid crystallization inhibitors include polyethylene glycols, polyoxyl glycerides, propylene glycol esters, diethylene glycol esters, glyceryl esters, polyoxyethylene sorbitan fatty acid esters, ethylene alkyl ethers, polyoxyethylene alkyl phenols, polyethylene glycol glycerol fatty acid esters, polyoxyethylene-polyoxypropylene block copolymers, polyoxyethylene glycerides, polyoxyethylene sterols, polyoxyethylene vegetable oils, and polyoxyethylene hydrogenated vegetable oils.

The amount of drug that can be incorporated in the film is generally from 0.01% to 50%, with preferred drug loading ranging from 1%-30% of the total weight of the film. Non-limiting examples of the pharmaceutically acceptable active ingredients that may be used in the invention include active ingredients that can exist in either or both amorphous and crystalline forms, such as hypnotics, sedatives, antiepileptics, awakening agents, psychoneurotropic agents, neuromuscular blocking agents, antispasmodic agents, antihistaminics, antiallergics, antidiarrhetics, cardiotonics, antiarrhythmics, diuretics, hypotensives, vasopressors, antitussive expectorants, thyroid hormones, sexual hormones, antidiabetics, antitumor agents, antibiotics and chemotherapeutics, central nervous system agent and narcotics.

The invention further provides, among other things, improved mechanisms to achieve a desired absorption rate, bioavailability or drug diffusion rate for at least one pharmaceutically active ingredient. While a rapid solubilization of the pharmaceutically active ingredient(s) is preferred, various desired solubilization profiles (i.e. plots of the quantity or quantities of the pharmaceutically active ingredient(s) absorbed by a liquid medium or mediums at particular time points) can be achieved by adjusting the properties of and procedures for producing the film dosage form. For example, the combination of an effectively stabilized particle size range (for example ca. 50-500 nm) exhibiting rapid solubilization, with a separately prepared, distinct, effectively stabilized particle size range (for example ca. 100-900 μm) demonstrating a decreased rate of solubilization of the same active relative to the plurality of particles exhibiting rapid solubilization, produces a dosage form that initially delivers the active rapidly followed by a slower rate of delivery that can be sustained over an effective period of time, preferably, twenty to forty five minutes.

The increase in solubility is due to a combination of an increase in the surface energy of the active particles and the stabilization of such. Factors which contribute to the improved stability of the active ingredient include a surprising and unforeseeable ability of the disclosed dosage forms to provide extensive physical and/or chemical protection to the active ingredient within the solid oral film.

The disclosed dosage forms use the dissolution properties of active molecules to effectively modulate their rate of absorption, bioavailability and/or diffusion rate when administered using oral film dosage forms. Since the dissolution properties are affected by particle size, the inclusion of two or more particle size distributions in an oral film formulation affects the active absorption rate and bioavailability. The smaller particle size distribution (SD) and the larger particle size distribution (LD) will be separated by a delta (DP) equal to the difference between LD D90 and SD D90 (DP=LD D90−SD D90).

However, the extent to which the absorption rate, bioavailability and diffusion rate are affected upon administration of such active when using oral films will depend on the intrinsic dissolution properties of the active molecule. Therefore, the present disclosure will distinguish between two groups of active, highly water soluble actives (HWSA) and poorly water soluble actives (PWSA).

Non limiting examples of PWSA are chosen from the group of Atorvastatin, Tadalafil, Cilostazol, Danazol Naproxen, Apomorphine Griseofulvin, Itraconazole, Buprenorphine, Loxapine, dutasteride, Montelukast, Dronabinol, miconazol, nitrazepam, propofol, diclofenac, Tetrahydrocannabinol.

Non limiting examples of HWSA include gentamicin, warfarin, metoprolol, naloxone, rizatriptan, colchicine, dextrometorphane, duloxetine, galantamine, methylphenidate, niacin, oxycodone, oxymorphone, prochloperazine, sildenafil, vitamin D, methotrexate and atenolol.

Since intrinsic dissolution properties affect the solubility of the active generally and most often invariably of the particle size (within useful particle size ranges), DP for PWSA will preferably be smaller than DP for HWSA. In other words, smaller DP will have a significant effect on PWSA, whereas larger DP will be required to observe a difference in the pharmacokinetic properties of HWSA. Yet, similar differences in particle size distribution (DP=50 μm) are useful for both PWSA and HWSA because of this DP (50 μm) affects the dissolution for both PWSA and HWSA (albeit to different extents). According to one embodiment, oral film dosage forms with PWSA have a first particle size range SD between about 5-100 μm, preferably between about 10 and 80 μm and a second particle size range LD between about 20-250 μm, preferably between about 30 to 120 μm with a DP of at least 10 μm, preferably at least 20 μm to effectively observe an absorption rate, bioavailability or drug diffusion rate modulated due to the dissolution rate of the particle once administered via oral film. The combination of multiple particle size distributions thus modulates the dissolution of the oral film to effectively yield a modulated absorption rate, bioavailability and/or diffusion rate based on the combination of result in different dissolution profile of such a small difference in active particle sizes.

For purposes of this disclosure, a drug substance is considered HIGHLY SOLUBLE (HWSA) when a 50 mg dose strength is soluble in 250 ml water over a pH range of 1 to 7.5. Conversely, poorly water soluble (PWSA) is herein defined as a drug in which the 50 mg dose strength is NOT soluble in 250 ml water over a pH range of 1 to 7.5.

Accordingly, the presently described oral film dosage forms comprise a plurality of distinct particle size distributions. The plurality of particle size distributions (e.g., bimodal, trimodal, etc) allows the modulation of absorption rate, bioavailability and diffusion rate of the active over time. The use of a plurality of particle size distributions is designed to modulate the rate of absorption and the bioavailability of active over a short period of time (as oppose to extended release formulations). This modulation is used to bridge the gap between exclusively fast release drug formulations in which no mechanism is present to delay or modulate bioavailability and rate of absorption, and extended release formulations purposefully designed to extend the rate of absorption and bioavailability over extended period of time typically lasting over periods as long as a day or more. The former (instant or rapid release) instantly releasing the active in the subject system whereas the latter allowing release over an extended period of time (i.e. several hours up to one day). There is thus a need for an oral film formulation that provides modulation of the absorption rate and area under the curve which is extended beyond immediate release without necessarily lasting for the extended periods of specific extended release formulations.

In certain preferred embodiments the plurality of distinct particle size distributions are incorporated into a stabilization system comprising at least one film forming polymer and at least one excipient, wherein the stabilization system inhibits changes to the distinct particle size distributions. Such stabilization systems preferably inhibit agglomeration of the active particles, crystallization (or recrystallization) of the active ingredient and/or provide other stabilizing effects on the particle size distributions of the active ingredient. Examples of such systems include those comprising crystallization inhibitors, as described herein, including primary crystallization inhibitors, and/or liquid crystallization inhibitors.

The dosage forms disclosed herein have a multimodal particle size distribution. A multimodal particle size distribution refers to a particle size distribution having a plurality of distinct modes or peaks (i.e., local maxima) and a distinct antimode or local minima between two distinct modes. Such particle size distributions can be determined using any of a variety of well-known particle size analysis techniques, such as laser diffraction, light scattering, and image analysis techniques. A distinct mode and antimode are those in which the difference between the mode and antimode cannot be attributable to error. The expression “D90” refers to the particle diameter that is greater than 90% of the active ingredient of a quantity of active ingredient having a particle size distribution. The multimodal particle size distributions of the disclosed dosage forms can be obtained by incorporating a first quantity of the active ingredient having a first particle size distribution (and an associated first D90) and a second quantity of the active ingredient having a second particle size distribution (and an associated second D90).

According to embodiments, oral films comprising one or more active ingredients present in a plurality of particle size distributions allow the modulation of the rate of absorption of the active using smaller sized particle distributions (varying the amount or loading of active present in SD) while also modulating the active bioavailability (area under the curve or “AUC”) in the bloodstream using larger particle size distributions of the active (by varying the amount of active present in LD). The smaller particles will start dissolving and vanishing before larger particles and have an impact on bioavailability and onset of action. In addition, including two or more particle size distributions (e.g., small, medium and large particles) will also permit extension of the availability of the drug over time and offer novel extended oral dosage form.

According to some embodiments, since this novel oral film dosage form and the method of formulating an oral film with a plurality of stabilized particle size distributions allows modulation of the rate of absorption and bioavailability of actives, it opens the possibility for actives with a small window of absorption (active which generally are not administered orally) to be suitable for oral administration via oral film dosage forms. Use of a multimodal particle size distribution modulates (increasing or decreasing) the absorption profile to prevent side effects generally associated with early Cmax peak in blood plasma concentrations (Cmax which occur below about 30 minutes). These oral film dosage forms allow the use of larger particle size within the film matrix to extend the time period wherein the plasma concentration is above the active agent efficacy level and modulate its bioavailability. In other words, larger particle size may be useful to modulate the shape of the plasma concentration curve. This modulation of the bioavailability is particularly useful for short half-life active ingredients (half-life of less than about 60 minutes) or active ingredients which are highly metabolized (i.e. superior to 40% of the active being orally metabolized). Thus the modulation of the bioavailability through incorporation of a second particle distribution in sufficient amount to increase the AUC over a period of time increases the period in which the active is present at an efficacy level in the bloodstream. This increased presence is due to the delayed and extended absorption of the larger size particles.

The rate of absorption can be modulated by varying the amount of smaller sized particle active (SD), the amount of larger sized particle (LD), or the ratio of SD/LD. The AUC can be modulated (increased or decreased) by the size or the spread of the larger particle size distribution LD. In addition, the AUC may be further modulated by including a third particle size distribution.

According to embodiments, the oral film formulation comprises an active having a plurality of preferably distinct, possibly overlapping particle size distributions having particle sizes (D50 or D90) between 500 nm to 200 μm, preferably between 5 and 100 The extent of the desired modulation in the absorption profile of the active thus affect the size difference between the average particle size distributions (D50 or D90). An oral dosage form comprising a PWSA includes at least two distinct particle size distributions (SD and LD). A first lot of active ingredient having a first particle size distribution SD, preferably the smallest particle size distribution, is added to the blend for dissolution (partial dissolution, total dissolution or salutation of the blend). Depending on the desired modulation of the bioavailability of the active, the active will be added to the blend and the blend will be adjusted for either partial dissolution (meaning that some undissolved active will remain present in the blend in the initial particle size), total dissolution (meaning that the active is substantially dissolved within the blend), or saturation (meaning that active is added to the blend until saturation of the blend). Then a second lot of active ingredient having a second particle size distribution LD is added to the blend already containing the first particle size distribution of active ingredient. The second lot LD (larger size particle) is generally added subsequently to have this second active particle size remain in the blend with its particle size preferably substantially undissolved and unaltered. PWSA may also be found in a film wherein the active is not solubilized at all in the film matrix, meaning that the final product comprises a plurality of particle size distributions but little to no active remaining dissolved within the film matrix subsequent to the drying phase. Other processes could include a simultaneous or joint addition of the two particle size distributions to the blend.

According to embodiments, PWSA contained oral films may be manufactured using aqueous or organic solvent blends whereas HWSA contained oral films will preferably be manufactured using organic solvents. The nature of HWSA renders these active prone to dissolution, thus somewhat hindering their ability to be sustained in an amorphous form and effectively preventing recrystallization of the HWSA once comprised in the dry film product. In addition, an effective particle distribution of multimodal character, is more conveniently generated from a film made from a blend within which the active ingredient had only little solubility. As such, HWSA oral films will have enhanced stability when manufactured using blending solvent in which the active is insoluble (i.e. non-aqueous solvent).

According to certain embodiments, the oral film having a plurality of particle size distributions is a single layer oral film in which the active is suspended. The single layer oral film thus comprises the active in a plurality of particle size distributions. The film is manufactured using a single blend comprising both particle size distribution suspended therein which is then cast and dried.

According to certain other embodiments, the oral film with a plurality of particle size distribution is a multilayer film in which a single layer of the multilayer oral film comprises the plurality of different particle size distributions of the active ingredient. This multilayer film can be made using a known method of making the film while using the method of making the single layer with multiple distribution size particle active as outlined herein. As such, according to these embodiments, the multilayer film incorporated a layer having an active (PWSA or HWSA or potentially both in case of a multiple active film) having at least two distinct particle size distributions.

According to some embodiments, different particle size distributions are present in distinct layers of a multilayer film product. Such a film is made using at least two blends. A first blend comprises a first particle size distribution (i.e. SD) and a second blend comprises a second particle size distribution (i.e. LD). Each blend may be coated as a separate layer and/or laminated with one another. Alternatively, a first layer may be coated (and dried) first (either SD or LD) while the second layer (either SD or LD) is coated on top of the previously coated layer. For multilayer film with more than two layers, the second layer (the layer having the second particle size distribution of active) may be coated on a backing or neutral layer positioned between the first and second layers.

The difference in the particle size distributions DP will be somewhat dependent on or likely to be affected by the solubility of the active ingredient in water and within the film matrix. The solubility of the at least partially permeable active correlatively affects the rate of absorption and bioavailability profile of the active ingredient within the body of a patient. DP will also depend on the dissolution properties of the active ingredient once orally administered to a patient.

Highly water soluble active ingredients (HWSA), as defined above refers to an active agent wherein the 50 mg dose strength is soluble in <250 ml water over a pH range of 1 to 7.5, are preferably present in a plurality of particle size distribution in such a way that the plurality of particle size distributions (SD and LD) covers a larger range of particle sizes than for the PWSA dosage forms, such that generally DP(HWSA)>DP(PWSA).

The basis for this difference in particle size ranges result from the fact that small particle size differences (small DP) have noticeable impacts on the dissolution of the PWSA whereas a smaller difference is observed when using the same DP for HWSA. The PWSA absorption upon oral administration is noticeably altered from a small DP (DP<25 μm). Therefore, HWSA oral film dosage forms having two of more particle size distributions (i.e. SD and LD) will generally have particle size distribution differences (DP) greater than 20 μm (DP>20 μm). According to another embodiment, an oral film dosage form comprising a HWSA, includes LD between 2*SD to 10*SD if SD D90>20 μm or LD between 5*SD to 10*SD if SD D90<20 μm, wherein both SD D90 and LD D90<250 μm.

Oral film dosage forms with PWSA can have particle size distributions (SD, LD, etc.) wherein the small differences between the first (SD) and second (LD) particle size distribution D90 will noticeably affect dissolution of the PWSA once orally administered. According to yet another embodiment, it is disclosed an oral film dosage form comprising a PWSA wherein DP<5*SD, preferably wherein DP is between 2*SD and 5*SD.

For actives having a high rate of absorption with a low bioavailability, an increase in the percentage of the second particle size distribution (LD) over the first particle size distribution (SD) would effectively increase bioavailability while not substantially impacting the rate of absorption which would be primarily affected by the smaller particle size distribution (SD). The larger size particle size would take longer than the smaller particle size to dissolve and generally be absorbed after the initial early Cmax peak, which in some cases will result in a bimodal absorption profile (i.e. two Cmax and two Tmax). This oral film formulation would effectively reduce the occurrence of side effects associated with the high early Cmax while allowing an increase in the bioavailability of the active ingredient.

Conversely, for active ingredients with high bioavailability but late onset, the present multimodal particle size distributions formulation is used for modulating the rate of absorption by increasing the quantity of the first smaller particle size distribution (SD) to accelerate or improve the rate of absorption. In addition, a subsequent even smaller particle size distribution is added SD2 for increasing the rate of absorption and improving the onset of the active (in which case SD2<SD<LD).

According to certain embodiments, the oral film dosage form that maintains a plurality of active ingredient particles in an effective particle size range to maintain reduced structural order, and/or improve solubility and bioavailability of the active, comprises at least one pharmaceutically active ingredient in the form of amorphous particles having a first and second particle size distribution, wherein the difference between the first (SD) and second (LD) D90 particle size is between 5 and 25 μm, preferably between 10 and 30 more preferably about 25 μm for PWSA.

The term “solid oral dosage form” as used herein refers to a physical form containing a predetermined amount of medication (i.e., dose) that may contain liquid or gaseous matter, but is primarily composed of solid matter having a higher Young's modulus and/or shear modulus than liquids.

The term “primary crystallization inhibitor” as used herein refers to a water soluble or water-dispersible, film-forming substance that is substantially chemically inert in the dosage form and is substantially chemically and biologically inert in the environment of use (e.g., buccal cavity), and has the effect of inhibiting growth and/or agglomeration of particles of a pharmaceutically active ingredient disposed in an oral film dosage form.

By employing suitable primary crystallization inhibitors, the particle growth and/or increase in the structural order of the pharmaceutically or therapeutically active ingredient can be inhibited during administration of the dosage form. Examples of primary crystallization inhibitors include polyvinyl pyrrolidone, polyethylene oxide and poloxamer. Film forming polymers that may be combined with the primary crystallization inhibitors include cellulose-derivatives, hydroxypropyl cellulose, hydroxyethyl cellulose, or hydroxypropylmethyl cellulose, carboxymethyl cellulose, and/or mixtures thereof. Other optional polymers include, carbomers, pregelatinized modified starch, polyvinyl alcohol, sodium alginate, polyethylene glycol, natural gums like xanthane gum, tragacantha, guar gum, acacia gum, arabic gum, carboxyvinyl copolymers. Suitable polymers may be employed in an amount ranging between 25% and 85% of the mass of the film dosage form.

The term plasticizer as used to describe and claim certain embodiments of the invention refers generally to a chemical entity that, when present, reduces the glass-transition temperature of amorphous polymers and the amorphous portion of semi-crystalline polymers. In particular, the present invention incorporates a plasticizer to impart flexibility, enhance elasticity and decrease brittleness. Preferred plasticizers include triacetin, citrate derivatives (such as triethyl, tributyl, acetyl tributyl, acetyl triethyl, trioctyl, acetyl trioctyl, trihexyl citrate, etc.), polyethylene glycol, glycerol and dibutyl sebacate. An amount of plasticizer that may be used is from about 2% to about 25% of the mass of the film dosage form.

The term “stabilized” as used herein refers to inhibition or retardation of changes of volume and/or loss of surface area, and/or increases in structural order of the plurality of active particles. More specifically, in the presence of certain macromolecules or polymers, the material shows an improved lifetime in an optimal particle size range, as characterized by reduced rate of agglomeration, increased structural order, crystallization and/or recrystallization of therapeutically active ingredient, providing a desired solubilization profile in a preferred liquid medium.

The term “penetration enhancer” as used herein refers to a substance that can increase buccal permeation of an active ingredient by enabling a transcellular route for transportation of the drug through the buccal epithelium. Certain non-limiting examples of pharmaceutically acceptable penetration enhancers include benzalkonium chloride, cetylpyridinium chloride, cyclodextrins, dextran sulfate, lauric acid/propylene glycol, menthol, oleic acid, oleic acid derivatives, polyoxyethylene, polysorbates, sodium EDTA, sodium lauryl sulfate, sodium salicylate.

The term “surfactant” as used herein refers generally to a chemical compound or substance that, when present in an effective amount, reduces the surface tension of a liquid and/or the interfacial tension between liquids.

The disclosed dosage forms may be prepared by first dispersing, suspending and optionally partially dissolving at least one therapeutically active ingredient and an optional antioxidant or antioxidants in at least one solvent. One or more liquid crystallization inhibitors are added, together with one or more plasticizers, optionally one or more penetrations enhancers and/or one or more optional surfactants. The film forming polymers are added and the mixture is kept under rotation until the film forming polymers have completely dissolved and a homogenous blend has been obtained. Optional ingredients such as flavors, sweeteners, taste maskers, antioxidants and colorants can be added at any time. It is preferred that the addition of other non-active ingredients is completed at an appropriate time as to minimize potential segregation, physical-chemical incompatibility or partial dissolution of the film forming polymers.

The final viscosity of the blend affects the film casting potential. Optimal viscosity ranges from 2000 centipoises to 90,000 centipoises. The final blend is transferred onto a surface of a suitable carrier material and dried to form a film. The carrier material must have a suitable surface tension in order to facilitate the homogenous distribution of the polymer solution across the intended coating width, without the formation of a destructive bond between the film and the carrier. Examples of suitable materials include siliconized polyethylene terephthalate film, non-siliconized polyethylene terephthalate film, non-siliconized paper, siliconized paper, polyethylene-impregnated kraft paper, and non-siliconized polyethylene film, siliconized polyethylene film. The transfer of the solution onto the carrier material can be performed using any conventional film coating equipment. A suitable coating technique would involve a knife-over-roll coating head. The thickness of the resulting film depends on the concentration of solids in the coating solution and on the gap of the coating head and can vary between 1 and 500 μm. Drying of the film may be carried out in a high-temperature air-bath using a drying oven, drying tunnel, vacuum drier, or any other suitable drying equipment. A desired dry film thickness of about 80-180 μm is typically targeted to facilitate the administration, drying and processing of the film. However, it is possible to make thinner and thicker films.

The following examples illustrate formulations, oral dosage forms and methods of preparing same in accordance with certain non-limiting aspects of the invention. All percentages in the examples are by weight unless otherwise indicated.

### Example 1

About 0.1 to about 5 g of a pharmaceutically active ingredient is dissolved in 110-290 ml of ethyl alcohol. To the resulting solution, 0.1 g of aspartame, 1.0 to 2.9 g of menthol/triacetine and 0.1-1.0 g of propylene glycol caprilate are added. Optionally, 0.1 to 1 g of polysorbate 80 and 0.1 to 1 g of polyoxyglyceride is added to the mixture. After one hour of stirring at high speed, 4 to 6 g of polyvinyl pyrrolidone and 0.1 to 0.5 g of pregelatinized modified starch are added and the mixture is stirred until homogenous. About 2.0 to 3.0 g of hydroxypropyl cellulose is added to the mixture. The blend is stirred for one hour before adding 0.02-0.08 g of colorant Yellow #6. Mixing is continued until a homogenous polymeric solution is obtained. About 25-35% of the solution is coated onto a suitable carrier material, for example non-siliconized, polyethylene-coated kraft paper, using conventional coating/drying equipment. Coating gap and web speed are adjusted to achieve a dry film thickness between 10 and 200 μm. The cast film is dried at a temperature of about 50 to 75° C. to achieve a desired effectively stabilized particle size range for immediate solubilization and the web speed is adjusted to completely remove the solvents from the film. The remaining 65-75% of the solution is cast on top of the previous film to achieve a dry film thickness from 40 μm to 60 μm, and dried at a temperature of 45-70° C. to achieve the desired effectively stabilized particle size range for a reduced rate of solubilization. The resulting film, with an intended average residence time of 30 minutes, is peeled off the carrier web and cut into pieces of a shape and size suitable for the intended use.

### Example 2

About 0.3 g of a pharmaceutically active ingredient is dissolved in 2 ml to 15.0 ml of ethyl alcohol and 40 to 56 ml of water. To the solution, 0.08 g of Talin, 0.15 g of aspartame, 2.0 g to 3.5 g of 10% menthol/triethyl citrate, 0.5 g to 1.5 g of polysorbate 80 were added and the resulting mixture is stirred at high speed for 1.5 hours. Optionally, the mixture can include 0.1 to 1.0 g of polyethyleneoxide and/or 0.2 g to 0.5 g of sodium EDTA. From 8.0 g to 10.0 g of polyvinyl pyrrolidone is added and the mixture is stirred for one more hour. From 2.0 g to 4.5 g of hydroxypropyl methyl cellulose type E15 was added to the mixture. Optionally, 0.5 g to 4.5 g of high molecular weight polyoxyethylene is added and the blend is stirred for one hour before adding 0.04 g of colorant Yellow #6 and 0.5 g of mint oil. Mixing is continued until a homogenous polymeric solution is obtained. The solution is coated onto a suitable carrier material, and dried at 45-75° C. for a time sufficient to remove the solvent.

### Example 3

About 2.8 g of a pharmaceutically active ingredient is dissolved in up to 4.5 ml of ethyl alcohol and from 51 ml to 75 ml of water. To the mixture, 0.5 g of ascorbic acid, 0.5 g of aspartame, 1.5 to 3 g of 14% menthol/triacetins, up to 0.5 g of polysorbate 20, and optionally 0.7 g of propylene glycol caprilate are added and the resulting mixture is stirred at high speed for 1 hour. From 6.0 to 8.0 g of polyvinyl pyrrolidone, 1.0 g to 2.5 g of polyethylene oxide 8000, and 0.2 g of pregelatinized modified starch are added to the mixture and stirred until it is homogenous. 1.0 g to 4.0 g of hydroxypropyl cellulose is added to the mixture. The blend is stirred for 1 hour before adding 0.04 g of colorant Blue#1. Mixing is continued until a homogenous polymeric solution is obtained. The solution is coated onto a suitable carrier material, and dried.

### Example 4

From 0.5 g to 0.7 g of two different pharmaceutically active ingredients are dissolved in 1.0 ml to 3.0 ml of acetone and 21 ml of water. To the resulting solution, 0.03 g of sucralose, 1.0 g to 2.0 g of triethyl citrate, 0.3 g of polysorbate 80, 0.5 g to 1.0 g of sodium phosphate dibasic and 0.1 g to 0.9 g of glyceryl mono oleate are added and the resulting mixture is stirred at high speed for 1 hour. From 4.0 g to 7.0 g of polyvinyl pyrrolidone and 0.2 g of pregelatinized modified starch are added and the mixture is stirred until homogenous. 1.0 g to 3.0 g of hydroxypropyl cellulose is added to the mixture. The blend is stirred for 3 hours before adding 0.02 g of colorant Yellow #5 and 0.2 g of vanilla flavor, mixed until homogenous, coated onto a suitable carrier material, and dried.

### Example 5

From 1.0 g to 2.0 g of pharmaceutically active ingredient and 0.1 g to 1.0 g of ascorbic acid are partially dissolved in a mixture of 45 ml of water, 4.2 g of 14% menthol/triacetine, and 2.0 g to 3.0 g of glyceryl mono oleate. To the suspension, 0.05 g of sucralose is added and the resulting mixture is stirred at high speed for 1 hour. From 3.0 g to 4.0 g of polyvinyl pyrrolidone and 0.1 g of pregelatinized modified starch are added and the resulting mixture is stirred until. homogenous. Optionally, from 2.0 g to 3.0 g of hydroxypropyl cellulose are added to the mixture. The blend is stirred for 1 hour before adding 0.01 g of colorant Blue #1. Mixing is continued until a homogenous polymeric solution is obtained. The solution is coated onto a suitable carrier material, and dried as described for Example 1.

### Example 6

A gastro-resistant granule preparation is made by combining a therapeutically active ingredient and a methacrylic polymer in a 2:1 to 1:2 weight to weight ratio, and optionally 1% to 5% of a disintegrant is placed in a jacketed bowl (i.e. mixer bowl) and mixed for homogenization. The jacket temperature is kept at about 65° C., the motor output is maintained at about 101-161 watts, and the mixer and chopper speeds are set to about 1500-1700 rpm. The jacket temperature is maintained at about 10° C. above the melting point range of the granulation liquid, which is obtained by heating a fatty alcohol or a mixture of fatty alcohols to about 55° C. Optionally, 1% to 10% of one or more surfactants by weight and/or 1% to 5% of disintegrants are added in the molten granulation liquid. The liquefied mixture is slowly added in portions to the preheated mixed powder blend, until the endpoint of the coating process is reached. After cooling down, the particle size of the granulated material is reduced to a dimension compatible with the thickness of the film to be cast. A suitable grinder is used to mill the granulated material. After screening, only the fraction under 0.5 mm is retained to be incorporated in the film blend.

A film blend is prepared by first dissolving one or more film forming polymers in pure water or in a mixture of water and 1% to 10% of organic solvents. The total concentration of polymers may be from about 20% to about 45% of the weight of the solution of which polyvinyl pyrrolidone is between 70% and 100% of the total weight of the polymers. Other ingredients added into the mixture include 2% to 5% of glyceryl mono oleate, 2% to 6% of tri-ethyl citrate, adequate amounts of taste maskers, sweeteners, flavors and colorants. The mixture is stirred until total dissolution of the polymers and homogenization of the ingredients is completed.

The viscosity of the blend is measured. Optimal values are from 30,000 to 45,000 centipoise. To the wet blend is added 1% to 50% w/w of the gastro resistant granules as described above. The resulting suspension is stirring for a minimum time sufficient to obtain a homogenous dispersion of the granules in the wet film blend. The solution is coated onto non-siliconized, polyethylene-coated kraft paper, using conventional coating/drying equipment. Coating gap and web speed is adjusted to achieve a dry film thickness between 100 and 300 μm. The drying temperature is 45-60° C. The resulting film is peeled off the carrier web and cut into pieces of a shape and size suitable for the intended use

### Example 7

From 1.0 g to 2.0 g of pharmaceutically active ingredient is dissolved in an acidified mixture of 10 ml of water, 0.2 g of triacetine and 0.1 g of polyethylene glycol. To the resulting solution, 0.1 g to 1.0 g of hydroxypropyl cellulose and 0.1 g to 2.0 g of a methacrylic acid copolymer demonstrating a pH-dependent solubility are added. The resulting suspension is stirred for 1 hour before adding 0.01 g of colorant Blue #1. The volume of water is adjusted to achieve a 20% solid weight content. Mixing is continued until a homogenous polymeric solution is obtained. The solution is spray dried onto sugar-starch pellets (e.g., SUGLETS®, 250-355 μm in size). To a pre-blended acidified solution containing 0.5 g of ascorbic acid, 0.5 g of aspartame, 1.5 g to 3 g of 14% menthol/triacetine, up to 0.5 g of polysorbate 20, and optionally 0.7 g of propylene glycol and/or 0.5 g caprilate, 6.0 g to 8.0 g of polyvinyl pyrrolidone, 1.0 g to 2.5 g of polyethylene oxide 8000, 0.2 g of pregelatinized modified starch and 0.04 g of colorant Blue#1 added. The spray-dried SUGLETS® pellets are suspended and mixed under high speed for 5-10 seconds or until homogenously distributed within the blend. The solution is coated onto a suitable carrier material 300 and 500 μm, and dried at a temperature of 55-80° C.

### Example 8

A formulation was developed for preparing solid oral film dosage forms for buccal and/or sublingual administration of a mixture containing tadalafil involving first the preparation of a tadalafil system that demonstrates increased aqueous solubility of the tadalafil for use in the preparation of the film using an aqueous solvent.

### Increased Tadalafil Solubilization Part A.

From 0.5 g to 0.7 g of tadalafil is dispensed in 20.0 ml to 30.0 ml of acetone. To the resulting solution, polyvinyl pyrrolidone is added slowly to a vortex at a mass required to precipitate the tadalafil and the polyvinyl pyrrolidone (1.0 to 5.0 g). The resulting precipitate is dried at 40° C. and then milled.

Preparation of Film in an Aqueous System Part B.

In 20-35 mL of water, 0.03 g of sucralose, 1.0 g to 2.0 g of triethyl citrate, 0.3 g of polysorbate 80, 0.5 g to 1.0 g of sodium phosphate dibasic and 0.1 g to 0.9 g of glyceryl mono oleate are added and the resulting mixture is stirred at high speed for 1 hour. Slowly add 4.0 g to 7.0 g of the product produced from part A containing the tadalafil demonstrating increased aqueous solubilization, and 0.2 g of pregelatinized modified starch are added and the mixture is stirred until homogenous. 1.0 g to 3.0 g of hydroxypropyl cellulose is added to the mixture. The blend is stirred for 3 hours before adding 0.02 g of colorant Yellow #5 and 0.2 g of vanilla flavor, mixed until homogenous, coated onto a suitable carrier material, and dried.

Modifications of the invention will occur to those skilled in the art and to those who make or use the invention. Therefore, it is understood that the embodiment(s) shown and described above are merely for illustrative purposes and not intended to limit the scope of the invention, which is defined by the following claims as interpreted according to the principles of patent law, including the doctrine of equivalents.

### Example 9

A formulation was developed for preparing solid oral film dosage forms for buccal and/or sublingual administration of a formulation containing the PWSA Atorvastatin, that utilizes two distinct API particle sizes to achieve the desired dissolution profile and consequent bioavailability for use in the preparation of film products using an aqueous/organic solvent.

Control Release Oral Films Using Particle Size Differential in Aqueous Blends.

To a 20-30 mL volume of water, 0.10 g sucralose, 0.08 g EDTA, 0.30 g starch, 0.050 g red iron oxide are added and mixed at 300 rpm. To this blend 1.80 g Atorvastatin with a D90 of 10 and 1.80 g Atorvastatin with a D90 of 50 μm are added and mixed at 300 rpm for 15 minutes, ensuring homogenous distribution. While mixing at 300 rpm, 4.00 g of pullulan is added. This is the main film forming polymer and generates the needed viscosity in the blend to maintain a homogenous distribution of the two particle sizes of API. The blend is stirred for 3 hours before adding 0.02 g of glycerin and 0.2 g of vanilla flavor, mixed until homogenous, coated onto a suitable carrier material, and dried.

### Control Release Oral Films Using Particle Size Differential in Organic Solvent Blends.

To a mixture of 20-30 mL volume of methylethyl ketone and 5-10 ml of iso-propyl alcohol, 0.20 g sucralose, 0.01 g BET, 0.08 g L-menthol, 0.13 g titanium dioxide are added and mixed at 300 rpm. To this blend 0.60 g of Rizatriptan with a D90 of 5 pm and 0.40 g of Rizatriptan with a D90 of 45 μm are added and mixed at 300 rpm for 15 minutes, ensuring homogenous distribution. Rizatriptan is used as an example of an API which is insoluble in an organic solvent. While mixing at 300 rpm, 3.00 g of hydroxypropyl cellulose is slowly added. The blend is stirred for 12 hours before adding 0.08 g of triacetine and mixed until homogenous. The blend is finally coated onto a suitable carrier material, and dried.

### Example 10

A formulation was developed for preparing solid oral film dosage forms for buccal and/or sublingual administration of a formulation containing the PWSA Atorvastatin, that utilizes two distinct API particle sizes to achieve the desired dissolution profile and consequent bioavailability for use in the preparation of film products using an aqueous/organic solvent. In this embodiment the film is designed as a bilayer or multilayer system where each respective layer contains a different size API.

Control Release Oral Films Using Particle Size Differential and Bilayer Films.

To a 20-30 mL volume of water, 0.10 g sucralose, 0.08 g EDTA, 0.30 g starch, 0.050 g red iron oxide are added and mixed at 300 rpm. To this blend 1.80 g Atorvastatin with a D90 of 15 μm is added and mixed at 300rpm for 15 minutes, ensuring homogenous distribution. While mixing at 300rpm, 4.00 g of pullulan is added. The blend is stirred for 3 hours before adding 0.02 g of glycerin and 0.2 g of vanilla flavor, mixed until homogenous, coated onto a suitable carrier material, and dried. A second blend with identical (or different) excipient composition is then prepared as the first and 1.80 g of Atorvastatin with a D90 of 4.5 μm is added and blended using the same conditions as the first mixture. This second blend is then cast on the first dried film layer (in certain embodiments it may also be laminated onto the first layer), to create a bilayer system in which each layer is contains an API with different particle sizes. In this example, the film product is envisioned to be placed on the oral mucosa or sublingually with the layer containing the smaller particle oriented down. In this way the first layer with the small particle size API can be rapidly absorbed initially, followed by the larger particles in the second exterior layer which will be absorbed over a longer period of time. In certain embodiments the layer containing the larger particle size may be directed downwards onto the mucosa. In other embodiments of this bilayer example the two films may be aqueous or organic solvent based, and can be combined by casting on top of a dried film or through lamination.

Control Release Oral Films Using Particle Size Differential and Multilayer Films.

To a 20-30 mL volume of water, 0.10 g sucralose, 0.08 g EDTA, 0.30 g starch, 0.050 g yellow iron oxide are added and mixed at 300 rpm. To this blend 1.80 g Atorvastatin with a D90 of 100 nm is added and mixed at 300 rpm for 15 minutes, ensuring homogenous distribution. While mixing at 300rpm, 4.00 g of pullulan is added. The blend is stirred for 3 hours before adding 0.02 g of glycerin and 0.2 g of vanilla flavor, and mixed until homogenous. This first blend is then cast and dried on a suitable liner material. As in the bilayer example, a second blend is then prepared using the same excipient formula but a D90 800nm particle size of active drug. This blend is then cast (or laminated) onto the first layer. Next a third blend is prepared similar to the two previous but containing a D90 of 500 μm, and this blend is then cast (or laminated) onto the bilayer. In this way creating an oral film with three layers, each containing a different API particle size. This process can be repeated as needed to create multilayer films where each layer contains a different particle size. In other embodiments of this multilayer example the films may be aqueous or organic solvent based, and can be combined by casting on top of a dried film or through lamination.

### Example 11

In another embodiment two different actives with two different particle sizes may be selected to be combined within a single film. Each active may require a particular particle size to achieve the desired dissolution and pharmacokinetics needed for synergistic therapeutic effects. In this embodiment samples would be prepared as described in Example 9, then the two different actives, the first with a SD D90 of 10 μm and a LD D90 of 50 μm and the second with a SD D90 of 5 μm and a LD D90 of 45 μm are added to the blend.

The combination of two different actives with two different particle sizes is not limited to a monolayer, and can be extended to both the bilayer and multilayer systems described here. This embodiment may also be extended to combinations of different multiple active drug molecules. In some embodiments different multiple actives may be included in the same film layer and then a second layer containing one or more different actives may be cast (or laminated) on top of the first layer.

What is claimed is:

1. An oral film dosage form that maintains a plurality of active ingredient particles in an effective particle size range to maintain reduced structural order, and/or modulate solubility and bioavailability of the active, comprising:
a. a pharmaceutically active ingredient in the form of amorphous particles having a first particle size distribution and a first D90 and a second particle size distribution having a second D90;
b. wherein a difference between the second D90 and the first D90 is between 1 to 4 times the first D90.

2. The oral film dosage form of claim 1, wherein the first D90 is between 5 and 80 μm.

3. The oral film dosage form of claim 1, wherein the first D90 and second D90 are between 5 to 100 μm.

4. The oral film dosage form of claim 1, wherein the pharmaceutically active ingredient is a poorly water soluble active.

5. The oral film dosage form of claim 1, wherein the active is selected from the group consisting of Tadalafil, Montelukast, Cilostazol, Danazol, Naproxen, Atorvastatin, Apomorphine Griseofulvin, Itraconazole, Buprenorphine, Loxapine, Dronabinol and Tetrahydrocannabinol.

6. The oral film dosage form of claim 5, wherein the difference between the first and second D90 is between 5 and 25 μm.

7. The oral film dosage form of claim 1, wherein the amorphous particles are capable of existing in crystalline form.

8. The oral film dosage form of claim 1, wherein the active ingredient particle size distribution is bimodal.

9. The oral film dosage form of claim 1, further comprising a third particle size distribution having a third D90, wherein the third D90 is larger than the second D90.

10. The oral film dosage form of claim 1, wherein the first D90 is between 500 nm and 15 μm and the second D90 is between 15 and 100 μm.

11. The oral film dosage form of claim 1, wherein the film is a multilayer oral film.

12. The oral film dosage form of claim 11, wherein the first and a second particle size distribution are in different layers.

13. An oral film dosage form that maintains a plurality of active ingredient particles in an effective particle size range to maintain reduced structural order, and/or modulate solubility and bioavailability of the active, comprising:
a. at least one pharmaceutically active ingredient in the form of amorphous particles having a first and a second particle size distribution, the first particle size distribution having a first D90 and the second particle size distribution having a second D90;
b. wherein a difference between the first D90 and the second D90 is between 1 to 9 times the first D90.

14. The oral film dosage form of claim 13, wherein the first D90 is smaller than 20 μm.

15. The oral film dosage form of claim 13, wherein the second D90 is between 2 to 10 times the first D90 and wherein the first D90 is equal or superior to 20 μm.

16. The oral film dosage form of claim 13, wherein the first and second D90s are between 5 to 200 μm.

17. The oral film dosage form of claim 13, wherein the pharmaceutically active ingredient is a highly water soluble active.

18. The oral film dosage form of claim 17, wherein the difference between the first and second D90 is equal or superior to 50 μm.

19. The oral film dosage form of claim 13, wherein the amorphous particles are capable of existing in crystalline form.

20. The oral film dosage form of claim 13, wherein the active particle size distribution is bimodal.

21. The oral film dosage form of claim 13, further comprising a third particle size distribution having a third D90, wherein the third D90 is larger than the second D90.

22. The oral film dosage form of claim 13, wherein the first D90 is between 5 and 25 μm and the second D90 is between 25 and 150 μm.

23. A film oral dosage form comprising a film forming polymer and a pharmaceutically active ingredient having a multimodal particle size distribution.
